SEQUENCE LISTING (2 sequences)

SEQ ID NO: 1 - DNA, 15 bp, artificial
  oligonucleotide
acaagagaacagaac

SEQ ID NO: 2 - DNA, 15 bp, artificial
  oligonucleotide
gttctgttctcttgt

(19) United States
(12) Patent Application Publication
(10) Pub. No.: US 2006/0166241 A1
(43) Pub. Date: Jul. 27, 2006

(54) Nucleic acid isolation unit and method using intercalator

(76) Inventors: Jeo-young Shim, Gyeonggi-do (KR); Kyu-youn Hwang, Incheon-si (KR); Joon-ho Kim, Gyeonggi-do (KR); Sung-ouk Jung, Gyeonggi-do (KR); Kyu-tae Yoo, Seoul (KR); Joon-shik Park, Gyeonggi-do (KR); Ji-na Namgoong, Gyeonggi-do (KR)

Correspondence Address: CANTOR COLBURN, LLP, 55 GRIFFIN ROAD SOUTH, BLOOMFIELD, CT, 06002, US

(21) Appl. No.: 11/325,834

(22) Filed: Jan. 5, 2006

(30) Foreign Application Priority Data
Jan. 25, 2005 (KR) 10-2005-0006575

(51) Int. Cl. C12Q 1/68; C12M 1/34

(52) U.S. Cl. 435/6; 435/287.2

(57) ABSTRACT

Provided are nucleic acid isolation unit and method. The method includes immobilizing an aromatic compound-containing nucleic acid intercalator on a solid support; contacting a first buffer solution containing a nucleic acid sample to be purified to the intercalator immobilized on the solid support to bind the intercalator with nucleic acids contained in the nucleic acid sample; cleaning the resultant structure where the nucleic acids are bound to the intercalator immobilized on the solid support; and eluting the nucleic acids with a second buffer solution.

### CROSS-REFERENCE TO RELATED PATENT APPLICATION

This application claims priority from Korean Patent Application No. 10-2005-0006575, filed on Jan. 25, 2005, in the Korean Intellectual Property Office, the disclosure of which is incorporated herein in its entirety by reference.

1. Field of the Invention

The present invention relates to a method of isolating and purifying nucleic acids using an intercalator.

2. Description of the Related Art

To perform amplification of desired targets after cell lysis, isolation of nucleic acids from a cell lysate containing proteins, etc. is required.

Currently known representative DNA purification techniques are described below.

For example, isolation of nucleic acids from a nucleic acid-bound silica solid by washing and elution with a buffer is most widely used [Boom et al., U.S. Pat. No. 5,234,809, 1993, Boom et al., J. Clin. Micrbiol. 28 (3), 495-503, 1990]. This method is based on the principle that nucleic acids of a nucleic acid-containing solution are bound to a surface of silica in the presence of a high concentration chaotropic salt such as GuHCl, Nal, or BuSCN, whereas they are separated from the nucleic acid-bound silica in the absence of a chaotropic salt or in the presence of a low concentration chaotropic salt. The precise determination of the interaction between two negatively charged materials, i.e., silica and nucleic acids, has not been carried out. However, the most persuasive explanation is that binding between silica and nucleic acids is mediated by a dehydration reaction [Meizak, K. A.; Sherwood, C. S.; Turner, R. F. B.; Haynes, C. A. Journal of Colloid and Interface Science 1996, 181, 635-644].

According to the explanation of the binding between silica and nucleic acids based on dehydration reaction, both silica and nucleic acids are electrically negatively charged and thus are hydrophilic. Thus, silica and nucleic acids are surrounded by water molecules in a common solution. However, the presence of a high concentration chaotropic salt reduces the number of water molecules surrounding silica and nucleic acids due to stronger hydrophilicity of the chaotropic salt than the silica and the nucleic acids, resulting in binding of the silica and the nucleic acids. When a salt concentration is changed after the binding of silica and nucleic acids, i.e., when the nucleic acid-bound silica is contacted to a solution containing low concentration or no chaotropic salt, elution of the nucleic acids from the silica occurs. Such reversible nucleic acid binding and elution can be efficiently used in purification and isolation of nucleic acids. However, a chaotropic salt is very toxic and acts as an inhibitor against a subsequent process such as PCR, and thus, must be removed after use. Furthermore, silica used for binding with negatively charged nucleic acids is also negatively charged, and thus may adversely affect PCR by electrostatic repulsive force. In addition, the above technique can be applied only to the isolation of high concentration DNAs.

There is also a technique of isolating and purifying nucleic acids by reversibly binding polyethyleneglycol (PEG) with nucleic acids [Hawkins et al., Nucleic Acids Res. 1995 (23):4742-4743]. This technique is based on solid phase reversible immobilization (SPRI). That is, a carboxyl group-coated solid, for example, a carboxyl group-coated magnetic bead is contacted to a high concentration PEG to form a PEG-immobilized magnetic bead, resulting in binding of nucleic acids to the PEG-immobilized magnetic bead. The nucleic acid-bound bead is separated and then subjected to nucleic acid elution in a low-concentration salt condition. This technique is also based on salt concentration adjustment. That is, nucleic acid binding occurs in a high concentration salt condition, whereas nucleic acid isolation occurs in a low concentration salt condition.

There is also a technique of binding nucleic acids to a solid support using a positively charged material, e.g., alumina, i.e., a technique of capturing nucleic acids using alumina coated on an inner wall of a microtube [U.S. Pat. No. 6,291,166, Xtrana]. According to this technique, the binding of nucleic acids to alumina is very strong and irreversible. Therefore, separation of the nucleic acids from the alumina is difficult, and thus, amplification of the nucleic acids occurs on the alumina. Since extraction, purification, and amplification of nucleic acids are performed in one container, inhibitors that may adversely affect a subsequent PCR process remain, thereby lowering the yield of PCR products. In addition, a NaOH buffer used for binding of alumina and nucleic acids is known as a PCR inhibitor.

In addition, a DNA purification kit which is commercially available from Qiagen can be used. According to this technique, nucleic acids are captured by anion exchange reaction in a buffer with a high salt concentration, washed with a buffer with a low salt concentration, and then amplified. This technique is the same as the above-described techniques in that the binding and elution of nucleic acids are performed by salt concentration adjustment.

As described above, common DNA isolation techniques capture nucleic acids based on charging properties of the nucleic acids or the use of an additional chemical substance (chaotropic salt, PEG, etc.), which renders DNA separation difficult. Furthermore, since DNA isolation requires several processes and the use of buffers with different compositions, common DNA isolation techniques cannot be easily applied to a LOC (Lab-On-a-Chip) or a LIP (Lab-In-Package).

In view of these problems, techniques using intercalators have been suggested. U.S. Pat. No. 4,921,805 discloses a method of capturing nucleic acids using ethidium bromide (EtBr) which is a widely known DNA intercalator dye. According to this method, EtBr is immobilized on a solid surface via a linker. Nucleic acid capturing occurs based on intercalation property into nucleic acids and positively charging property of EtBr. However, the binding of nucleic acids with EtBr is very strong and thus separation of nucleic acids is difficult. To separate nucleic acids, an alkaline condition is required. For this, 0.5M NaOH must be used. However, since NaOH is known as a PCR inhibitor, the yield of PCR products may be lowered. In addition, since EtBr is known as a very strong toxic substance, additional costs and time for EtBr disposal are required.

### SUMMARY OF THE INVENTION

The present invention provides a method of isolating and purifying nucleic acids at high efficiency under a mild condition that does not affect a subsequent amplification process.

According to an aspect of the present invention, there is provided a nucleic acid isolation unit including: a solid support; a polymer layer coated on the solid support; and a nucleic acid intercalator including an aromatic compound immobilized on the polymer layer.

The solid support may be in the form of a plate or a bead.

The polymer layer may include at least one functional group. The functional group has high chemical reactivity and may be a hydroxy group, an amino group, a thiol group, a carboxy group, an alkoxy group, or a formyl group.

A polymer of the polymer layer is not particularly limited provided that it is a polymer material with a polymer structure. The polymer may be polysilane, polyalcohol, polyvinyl, or polystyrene.

The nucleic acid intercalator may be covalently bound to the polymer layer.

The nucleic acid intercalator is not particularly limited provided that it can be intercalated into double-stranded DNAs or attached to single-stranded DNAs by base-stacking. The nucleic acid intercalator may be a substituted or unsubstituted aromatic compound of 10 to 100 carbon atoms. The aromatic compound may have 2 to 6 benzene rings and the benzene rings may be attached to each other as a pendant group or be partially or wholly fused. One or more hydrogen atoms on a fused or unfused aromatic compound may be substituted by a substituent such as a halogen atom, a hydroxy group, an amino group, a nitro group, a cyano group, a substituted or unsubstituted alkyl group of 1-12 carbon atoms, a substituted or unsubstituted alkenyl group of 2-12 carbon atoms, a substituted or unsubstituted alkoxy group of 1-12 carbon atoms, etc. Examples of the substituted or unsubstituted aromatic compound include naphthalene, anthracene, phenanthrene, pyrene, chrysene, tetracene, benzofuran, indole, benzothiophene, carbazole, quinoline, and benzoquinone.

According to another aspect of the present invention, there is provided a nucleic acid isolation method using an intercalator including: immobilizing an aromatic compound-containing nucleic acid intercalator on a solid support; contacting a first buffer solution containing a nucleic acid sample to be purified to the intercalator immobilized on the solid support to bind the intercalator with nucleic acids contained in the nucleic acid sample; cleaning the resultant structure where the nucleic acids are bound to the intercalator immobilized on the solid support; and eluting the nucleic acids with a second buffer solution.

The nucleic acid intercalator may be the above-described aromatic compound.

The nucleic acids may be double-stranded DNAs or single-stranded DNAs.

The first buffer solution may have a salt concentration of 0.1 to 0.3M. The second buffer solution may have a salt concentration of 0.5 to 2M. The second buffer solution may be a nucleic acid amplification buffer, in particular, a PCR buffer. The second buffer solution may have a temperature of 70 to 100° C.

The cleaning may be performed using a phosphate-containing cleaning buffer.

### BRIEF DESCRIPTION OF THE DRAWINGS

The above and other features and advantages of the present invention will become more apparent by describing in detail exemplary embodiments thereof with reference to the attached drawings in which:

FIG. 1 is a diagram illustrating nucleic acid capturing according to a method of the present invention;

FIG. 2 is a view illustrating relative fluorescence intensities with respect to temperature for elution of oligonucleotides in distilled water and alkaline conditions according to a method of the present invention;

FIG. 3 is a view illustrating relative fluorescence intensities with respect to temperature for elution of oligonucleotides in 10×SSPET buffer and 10×PCR buffer according to a method of the present invention;

FIG. 4 is a graph illustrating relative fluorescence intensities with respect to time for capturing of bacterial DNAs according to a method of the present invention;

FIG. 5 is a view illustrating relative fluorescence intensities with respect to temperature for elution of bacterial DNAs in 10×TE buffer and 10×PCR buffer according to a method of the present invention; and

FIG. 6 illustrates an example of a substrate embodying a method of the present invention.

### DETAILED DESCRIPTION OF THE INVENTION

Hereinafter, the present invention will be described in more detail.

According to a method of the present invention, isolation and purification of nucleic acids can be performed without using a toxic substance such as a chaotropic salt or EtBr, unlike a conventional technique. Furthermore, unlike a conventional technique which cannot easily isolate nucleic acids due to nucleic acid capturing through charge-charge interactions, nucleic acid capturing is performed using an uncharged substance and separation of nucleic acids is easily performed by adjusting an elution condition, thereby remarkably increasing the yield of PCR products.

To accomplish the above effects, in the present invention, nucleic acids are captured using an uncharged nucleic acid intercalator and eluted in high efficiency in an appropriate elution condition. The eluted nucleic acids can be immediately used in subsequent PCR amplification. According to the present invention, the purification and PCR of nucleic acids can be performed on the same substrate.

Unlike a conventional technique in which the binding of nucleic acids occurs at a high salt concentration and the elution of the nucleic acids occurs at a low salt concentration, according to the present invention, the binding of nucleic acids can be performed at a low salt concentration and the elution of nucleic acids can be more efficiently performed at a high salt concentration.

The present invention provides a nucleic acid isolation unit including a solid support; a polymer layer coated on the solid support; and a nucleic acid intercalator including an aromatic compound immobilized on the polymer layer.

The solid support is not particularly limited but may be made of glass or plastic. For example, the solid support may be made of silicone, glass, silica, diamond, quartz, alumina, a metal such as platinum, aluminum, or tungsten, polyester, polyamide, polyimide, acryl, polyether, polysulfone, or fluoropolymer. The form and size of the solid support are not particularly limited. For example, the solid support may be a flat board, a wafer, a fiber, a bead, a particle, a chain, a gel, a sheet, a sphere, a pad, a pillar, a slide, a thin film, or a plate. The solid support may also be a capillary tube, a channel, a membrane, a test tube, a column, a pin, or a glass fiber. A bead or a plate is preferable.

The solid support is coated with the polymer layer. The polymer layer is linked with the intercalator to immobilize the intercalator.

The polymer that can be used for the polymer layer is not particularly limited provided that it is a polymer material having a polymer structure. For example, the polymer may be polysilane, polyvinyl, or polystyrene.

Preferably, the polymer layer has at least one functional group. The functional group is not particularly limited provided that it facilitates binding between the polymer layer and the intercalator. The functional group may be a hydroxy group, an amino group, a thiol group, a carboxy group, an alkoxy group, or a formyl group.

Preferably, the intercalator is linked to the polymer layer by a covalent bond.

Generally, the term “intercalator” refers to a material capable of being intercalated into the base pairs of double-stranded DNAs. Various types of materials that can be used as intercalators are well known in the art.

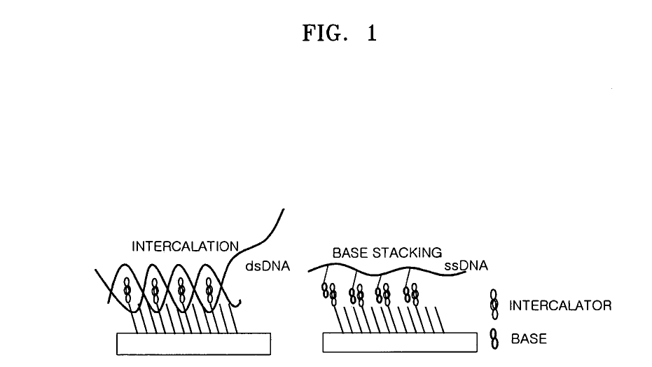

An intercalator that can be used in the nucleic acid isolation unit of the present invention is preferably a material capable of being intercalated into both double-stranded DNAs and single-stranded DNAs. FIG. 1 illustrates the binding of an intercalator of the present invention with a double-stranded DNA or a single-stranded DNA. Referring to FIG. 1, DNA capturing occurs by intercalation of the intercalator into the base pairs of the double-stranded DNA. With respect to the single-stranded DNA, bases of the single-stranded DNA are linked to the intercalator by base-stacking. Therefore, isolation of all DNAs is possible.

Preferably, an intercalator that can be used herein is an uncharged intercalator to enable the elution of nucleic acids, which is an important feature of the present invention. The uncharged intercalator will be described later in more detail in a nucleic acid purification method.

An intercalator satisfying all the above-described requirements may be an aromatic compound, preferably a substituted or unsubstituted aromatic compound of 10 to 100 carbon atoms. Preferably, the aromatic compound contains 2 to 6 benzene rings. The benzene rings may be attached to each other as a pendant group or may be fused partially or wholly. One or more hydrogen atoms on a fused or unfused aromatic compound may be substituted by a substituent such as a halogen atom, a hydroxy group, an amino group, a nitro group, a cyano group, a substituted or unsubstituted alkyl group of 1-12 carbon atoms, a substituted or unsubstituted alkenyl group of 2-12 carbon atoms, a substituted or unsubstituted alkoxy group of 1-12 carbon atoms, etc. Examples of the substituted or unsubstituted aromatic compound include naphthalene, anthracene, phenanthrene, pyrene, chrysene, tetracene, benzofuran, indole, benzothiophene, carbazole, quinoline, and benzoquinone.

The present invention also provides a nucleic acid isolation method including:
immobilizing an aromatic compound-containing nucleic acid intercalator on a solid support;
contacting a first buffer solution containing a nucleic acid sample to be purified to the intercalator immobilized on the solid support to bind the intercalator with nucleic acids contained in the nucleic acid sample;
cleaning the resultant structure where the nucleic acids are bound to the intercalator immobilized on the solid support; and
eluting the nucleic acids with a second buffer solution.

The nucleic acid isolation method will now be described in more detail.

There are no particular limitations to the operation of immobilizing the aromatic compound-containing intercalator on the solid support provided that the intercalator can be immobilized on the solid support. Preferably, the above-described nucleic acid isolation unit can be utilized for the immobilization of the intercalator on the solid support.

The aromatic compound-containing intercalator that can be used in the method of the present invention is as defined in the above. That is, it is preferable that the aromatic compound-containing intercalator is an uncharged aromatic compound capable of binding with both a double-stranded DNA and a single-stranded DNA. Since the intercalator of the present invention is uncharged, the binding of it with a nucleic acid occurs through base-stacking, as shown in FIG. 1.

According to a common nucleic acid capturing technique using a charged intercalator, nucleic acids are linked to the charged intercalator via an ionic bond (10 Kcal/mol), which makes it difficult to separate the nucleic acids during a subsequent elution process. However, according to the present invention using an uncharged intercalator, nucleic acids are linked to the uncharged intercalator through a Van der Waals force (<1 Kcal/mol) only, and thus, a binding energy required to separate the nucleic acids from the uncharged intercalator is small, thereby leading to efficient separation of the nucleic acids from the intercalator during a subsequent elution process.

The weaker binding force of nucleic acids with an uncharged intercalator can be compensated for by sufficiently increasing a surface area or the amount of the intercalator. However, it is difficult to solve the problem that an irreversible interaction between nucleic acids and a charged intercalator renders separation of the nucleic acids from the intercalator difficult, like in a common nucleic acid isolation technique, resulting in a remarkable reduction in yield of PCR products. In this regard, according to the nucleic acid isolation method of the present invention, the product yield of a subsequent PCR process is significantly increased relative to a common technique.

In the operation of contacting the first buffer solution containing the nucleic acid sample to the intercalator immobilized on the solid support, the nucleic acids contained in the nucleic acid sample are captured onto the intercalator. A pH condition is not limited. However, the first buffer solution may be a buffer solution containing NaCl or phosphate, preferably a SSPET buffer or a phosphate buffer.

Preferably, the first buffer solution has a salt concentration of 0.1 to 0.3M. Under these conditions, a high nucleic acid capturing efficiency can be accomplished.

The solid support on which the nucleic acids are captured is washed before elution of the nucleic acids. The cleaning may be performed using a cleaning buffer containing NaCl or phosphate.

The operation of eluting the nucleic acids is performed using the second buffer solution. A pH condition is not limited but the second buffer solution may be a buffer solution containing NaCl, Tris-HCl, or EDTA. A PCR buffer containing Tris-HCl or EDTA may be used. A PCR buffer that can be used as a PCR solution has an advantage when used as the second buffer solution in that eluted nucleic acids can be directly used for PCR amplification. A PCR buffer as used herein contains 250mM NaCl, 50mM Tris-HCl, or 10mM MgCl₂.

Preferably, the second buffer solution is a 10-times or more concentrated TE buffer or PCR buffer having a high salt concentration for high ionic strength. The present inventors found that in performing a nucleic acid isolation method of the present invention, the elution of nucleic acids at a high salt concentration was more efficient than a common nucleic acid elution at a low salt concentration.

Thus, it is preferable that the second buffer solution has a salt concentration of 0.5 to 2M.

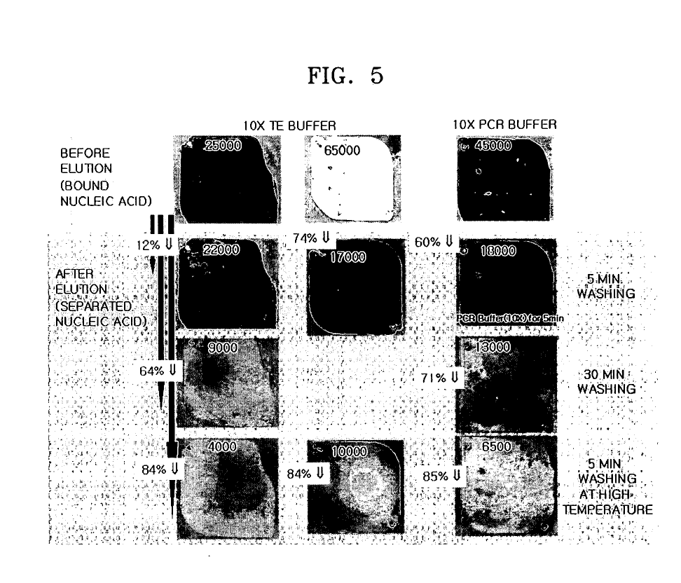

Furthermore, the elution of nucleic acids is more efficient at high temperature (see FIG. 5). In this regard, the temperature of the second buffer solution is preferably is in the range from 70 to 100° C., more preferably from about 85 to 95° C., and most preferably about 90° C.

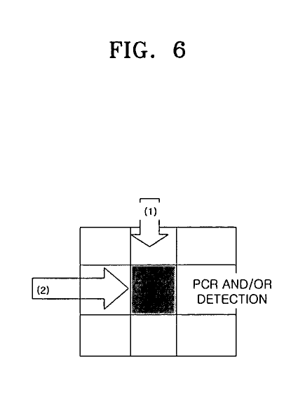

As described above, the use of a PCR buffer as the second buffer solution according to the nucleic acid isolation method of the present invention enables in-situ PCR reaction and an exemplary diagram thereof is illustrated in FIG. 6.

FIG. 6 is a plan view illustrating an example of a substrate for nucleic acid isolation according to an embodiment of the present invention. Referring to FIG. 6, the substrate is formed with a cruciform chamber and only a center of the chamber is immobilized with an intercalator. First, a nucleic acid sample is injected into the chamber in the direction of arrow (1) to capture nucleic acids on the intercalator, followed by washing. Then, a PCR buffer is injected into the chamber in the arrow direction of (2) to elute the nucleic acids captured on the intercalator. The eluted nucleic acids can be directly used for PCR.

The substrate shown in FIG. 6 can be modified provided that an object of the present invention can be accomplished.

Hereinafter, the present invention will be described more specifically with reference to the following examples.

### EXAMPLES

### Example 1

In this Example, silicone substrates on which a SiO₂ layer was formed to a thickness of 1,000 Å were used. Coupling agents (GAPS) were attached to the silicone substrates and then intercalators were immobilized onto the silicone substrates. The immobilization of the intercalators onto the silicone substrates was identified by a fluorescent scanner. Then, complementary oligonucleotides to the intercalators were captured on the intercalator-immobilized substrates by hybridization. The intercalation of the intercalators into the oligonucleotides was identified by a fluorescent scanner.

1-1. Attachment of Coupling Agents (GAPS) To Silicone Substrates

First, silicone substrates were carefully cleaned prior to a surface treatment. The cleaning was performed with pure acetone and water. Then, organic contaminants were removed from the silicone substrates using a piranha solution (1:3 mixture of hydrogen peroxide and sulfuric acid). Finally, the substrates were washed with abundant water and acetone and then dried. The cleaning was performed in a wet station used in a semiconductor fabrication process, the removal of the organic contaminants with the piranha solution was performed in a sulfuric acid bath, and the washing was performed using a QDR (Quick Dry Rinse) process. The washing was performed after fixing the substrates to a silicone wafer carrier made of Teflon. The drying was performed using a spin dryer.

Immediately after the washing, the substrates were spin-coated with a 20% (v/v) solution of GAPS (gamma (γ)-aminopropyltriethoxysilane) in ethanol or a 20% (v/v) solution of GAPDES(gamma-aminopropyldiethoxysilane) in ethanol. The spin coating was performed using a spin coater (Model CEE 70, CEE) as follows: initial coating at a rate of 500 rpm/10 sec. and a main coating at a rate of 2,000 rpm/10 sec. When the spin coating was completed, the substrates were fixed to a Teflon wafer carrier and cured at 120° C. for 40 minutes. The cured substrates were dipped in water for 10 minutes, ultrasonically washed for 15 minutes, again dipped in water for 10 minutes, and dried. The drying was performed using a spin dryer. The dried substrates were cut into squares or rectangles for the following experiments. All the experiments were performed in a clean room-class 1000 in which most dust particles were sufficiently removed.

1-2. Intercalator Immobilization

The silanized substrates prepared in 1-1 were coated with intercalators. Pyrenes were used as the intercalators and substrate coating with the intercalators was performed by dipping.

In detail, first, 1-pyrenebutyric acid N-hydroxysuccinimide ester (hereinafter, simply referred to as “pyrene”) was dissolved in a methylene chloride solution to obtain a dipping solution (0.5 g pyrene/200 ml+0.1 ml triethylamine). The dipping solution and the substrates were placed in a reaction chamber and incubated at room temperature for 5 hours. After the reaction was completed, the substrates were removed from the dipping solution, cleaned with methylene chloride (×3, 10 minutes for each) and ethanol (×3, 10 minutes for each), and dried.

Pyrenes immobilized on the substrates were quantified using a fluorescent scanner (GenePix 4000B, Axon). Scanning was performed at 532 nm and fluorescence intensity was measured at 570 nm. As a result, it was observed that immobilization of pyrenes on the substrates sufficiently occurred.

1-3. Capturing of Oligonucleotides On Intercalator-Immobilized Substrates

Hybridized oligonucleotides were captured on the pyrene-immobilized substrates prepared in 1-2 as follows.

First, patches for incorporation of an oligonucleotide solution were attached to the pyrene-immobilized substrates.

Then, Cy5-labelled oligonucleotides (5′-ACA AGA GAA CAG AAC-3′) (SEQ ID NO: 1) (400pM) and complementary oligonucleotides thereof (5′-GTT CTG TTC TCT TGT-3′) (SEQ ID NO: 2) (40nM) were hybridized in a 3× phosphate buffer for one hour.

About 60 μl of the hybridized oligonucleotides were added to the pyrene-immobilized substrates and incubated for one hour so that the pyrenes immobilized on the substrate were intercalated into the oligonucleotides. The intercalation of the pyrenes into the oligonucleotides was performed at room temperature. After the reaction was completed, the substrates were cleaned with a 3×SSPET (Sodium Phosphate+EDTA+Triton) buffer and then the intercalation amount of the pyrenes into the oligonucleotides was measured in PMT 700 using a fluorescent scanner (GenePix 4000B, Axon).

1-4. Elution of Oligonucleotides

1-4-1. Elution of Oligonucleotides With Distilled Water Or Carbonate Buffer

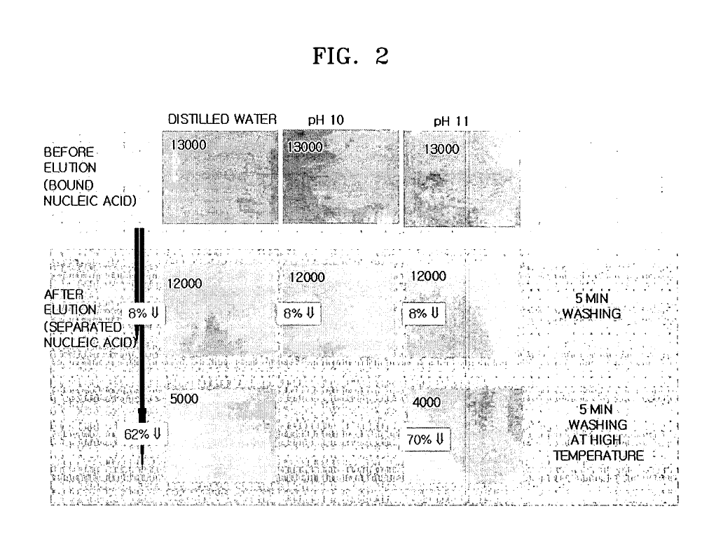

The oligonucleotide-capturing pyrene-immobilized substrates prepared in 1-3 were cleaned with distilled water, a carbonate buffer (NaCl, NaHCO₃, pH 10), and a carbonate buffer (NaCl, NaHCO₃, pH 11) (5 minutes for each) at room temperature, and then it was determined whether the oligonucleotides were eluted. The results, represented by fluorescence intensities before and after the elution, are shown in FIG. 2

Referring to FIG. 2, 8% of the oligonucleotides were eluted after being cleaned under distilled water and alkaline conditions for 5 minutes, whereas 62% and 70% of the oligonucleotides were respectively eluted after being cleaned at 90° C. or more under distilled water and alkaline conditions for 5 minutes. These results show that elution of nucleic acids scarcely occurs at high pH and low ionic strength.

1-4-2. Elution of Oligonucleotides With 10×SSPET Buffer Or PCR Buffer

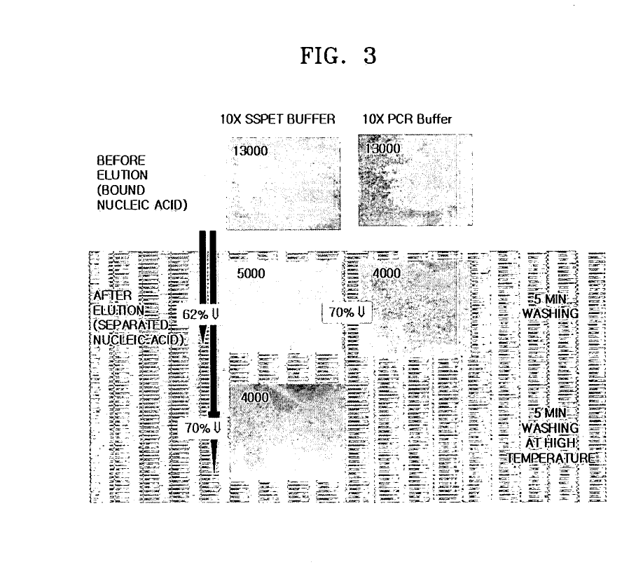

The oligonucleotide-capturing pyrene-immobilized substrates prepared in 1-3 were cleaned with a 10×SSPET buffer solution and a 10×PCR buffer solution (mainly containing a TE (250 mM NaCl+10 mM Tris-HCl) buffer, Qiagen) for 5 minutes (for each) and the results are shown in FIG. 3. Referring to FIG. 3, about 60% and 70% of the oligonucleotides were respectively eluted after cleaning with the 10×SSPET buffer solution and the 10×PCR buffer solution. In addition, after the oligonucleotide-capturing pyrene-immobilized substrates were cleaned with a 10×SSPET buffer solution at 90° C. or more for 5 minutes, about 70% of the oligonucleotides was eluted. As described above, the degree of the elution was evaluated by measuring fluorescence intensities before and after the elution.

It can be seen from the above results that the elution of oligonucleotides can be performed in high ionic strength and high temperature conditions and is not significantly affected by pH, etc., and desired elution can be accomplished in a 10× or more buffer condition, i.e., at a high salt concentration and high temperature.

### Example 2

2-1. Capturing of Bacterial DNAs Onto Intercalator-Immobilized Substrates

Capturing of bacterial DNAs onto the pyrene-immobilized substrates prepared in 1-2 of Example 1 was performed as follows. E. Coli DNAs of about 210 bp were used as the bacterial DNAs, and the bacterial DNAs were labeled with Cy5 fluorescence.

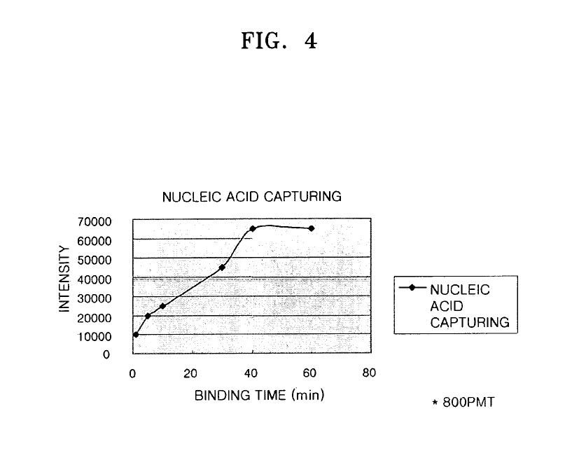

First, the Cy5-labelled bacterial DNAs (about 200mer in length) were dissolved in a 3× phosphate buffer and adjusted to 17nM. 60 μl of the bacterial DNA solution was added to five groups of the pyrene-immobilized substrates and incubated at room temperature for 1, 5, 10, 30, and 40 minutes, respectively, so that the capturing of the bacterial DNAs onto the pyrene-immobilized substrates occurred. After the reaction was terminated, the five substrate groups were cleaned with a 3×SSPET buffer and the amount of intercalation of the pyrenes into the bacterial DNAs was measured using the same fluorescence scanner as mentioned above. Kinetic experiments were performed to measure the actual amount of the captured DNAs and the experimental results are shown in FIG. 4.

Referring to FIG. 4, it can be seen that sufficient capturing of the bacterial DNAs occurred after the incubation for 30 minutes or more.

2-2. Elution of Bacterial DNAs

2-2-1. Elution of Bacterial DNAs With 10×TE Buffer

The bacterial DNA-capturing pyrene-immobilized substrates obtained by incubating the bacterial DNAs and the pyrene-immobilized substrates for 10 and 40 minutes among the bacterial DNA-capturing pyrene-immobilized substrates prepared in 2-1 were cleaned with a 10×TE (Tris+EDTA) buffer solution for 5 minutes and the results are shown in FIG. 5. Referring to FIG. 5, elution of about 70% of the bacterial DNAs was observed.

In addition, the bacterial DNA-capturing pyrene-immobilized substrates obtained by incubating the bacterial DNAs and the pyrene-immobilized substrates for 10 and 40 minutes were cleaned with a 10×SSPET buffer solution at 90° C. or more for 5 minutes. As a result, about 80% of the bacterial DNAs was eluted (see FIG. 5).

The degree of the elution was evaluated by measuring fluorescence intensities before and after the elution.

2-2-2. Elution of Bacterial DNAs With 10×PCR Buffer

The bacterial DNA-capturing pyrene-immobilized substrates prepared in 2-1 were cleaned with a 10×PCR buffer solution for 5 minutes. As a result, about 60% of the bacterial DNAs was eluted (see FIG. 5). In addition, the cleaning of the bacterial DNA-capturing pyrene-immobilized substrates with a PCR buffer solution at 90° C. or more for 5 minutes resulted in elution of about 80% of the bacterial DNAs (see FIG. 5). The degree of the elution was evaluated by measuring fluorescence intensities before and after the elution.

As described above, it can be seen that bacterial DNAs captured onto a pyrene-immobilized substrate are easily eluted under a high ionic strength condition, i.e., a 10× or more buffer condition and a high temperature condition.

According to the present invention, isolation and purification of nucleic acids can be performed without using a toxic substance such as a chaotropic salt and EtBr, unlike in a conventional technique. Furthermore, nucleic acid capturing is performed using an uncharged substance and elution is performed in an appropriate elution condition to solve the problem of a conventional technique in which capturing of nucleic acids onto a charged substance renders separation of the nucleic acids from the charged substance difficult. Therefore, the product yield of a subsequent PCR process can be remarkably increased.

What is claimed is:

1. A nucleic acid isolation unit comprising:
a solid support;
a polymer layer coated on the solid support; and
a nucleic acid intercalator comprising an aromatic compound immobilized on the polymer layer.

2. The nucleic acid isolation unit of claim 1, wherein the solid support is in the form of a plate or a bead.

3. The nucleic acid isolation unit of claim 1, wherein the polymer layer comprises at least one functional group selected from the group consisting of a hydroxy group, an amino group, a thiol group, a carboxy group, an alkoxy group, and a formyl group.

4. The nucleic acid isolation unit of claim 1, wherein the polymer layer comprises at least one polymer selected from the group consisting of polysilane, polyalcohol, polyvinyl, and polystyrene.

5. The nucleic acid isolation unit of claim 1, wherein the nucleic acid intercalator is covalently bound to the polymer layer.

6. The nucleic acid isolation unit of claim 1, wherein the nucleic acid intercalator is a substituted or unsubstituted aromatic compound of 10 to 100 carbon atoms.

7. The nucleic acid isolation unit of claim 6, wherein the aromatic compound has 2 to 6 benzene rings and the benzene rings are attached to each other as a pendant group or are partially or wholly fused.

8. The nucleic acid isolation unit of claim 6, wherein the aromatic compound is at least one selected from the group consisting of naphthalene, anthracene, phenanthrene, pyrene, chrysene, and tetracene.

9. A nucleic acid isolation method using an intercalator comprising:
immobilizing an aromatic compound-containing nucleic acid intercalator on a solid support;
contacting a first buffer solution containing a nucleic acid sample to be purified to the intercalator immobilized on the solid support to bind the intercalator with nucleic acids contained in the nucleic acid sample;
cleaning the resultant structure where the nucleic acids are bound to the intercalator immobilized on the solid support; and
eluting the nucleic acids with a second buffer solution.

10. The nucleic acid isolation method of claim 9, wherein the nucleic acid intercalator is a substituted or unsubstituted aromatic compound of 10 to 100 carbon atoms.

11. The nucleic acid isolation method of claim 10, wherein the aromatic compound has 2 to 6 benzene rings and the benzene rings are attached to each other as a pendant group or are partially or wholly fused.

12. The nucleic acid isolation method of claim 10, wherein the aromatic compound is at least one selected from the group consisting of naphthalene, anthracene, phenanthrene, pyrene, chrysene, and tetracene.

13. The nucleic acid isolation method of claim 9, wherein the nucleic acids are double-stranded DNAs or single-stranded DNAs.

14. The nucleic acid isolation method of claim 9, wherein the first buffer solution has a salt concentration of 0.1 to 0.3M.

15. The nucleic acid isolation method of claim 9, wherein the second buffer solution has a salt concentration of 0.5 to 2M.

16. The nucleic acid isolation method of claim 9, wherein the second buffer solution is a nucleic acid amplification buffer.

17. The nucleic acid isolation method of claim 16, wherein the nucleic acid amplification buffer is a PCR (Polymerase Chain Reaction) buffer.

18. The nucleic acid isolation method of claim 9, wherein the second buffer solution has a temperature of 70 to 100° C.
